(19) United States
(12) Patent Application Publication
(10) Pub. No.: US 2012/0123547 A1
(43) Pub. Date: May 17, 2012

(54) INTERVERTEBRAL DISC STRAIN-RELIEF SUPPORT

(76) Inventor: Ulrich Holzwarth, Eckental (DE)

(21) Appl. No.: 13/262,431

(22) PCT Filed: Jan. 14, 2010
(86) PCT No.: PCT/EP2010/050420

§ 371 Date: Feb. 7, 2012

(30) Foreign Application Priority Data
Jan. 21, 2009 (CH) 91/2009

(51) Int. Cl. A61F 2/44

(52) U.S. Cl. 623/17.16

(57) ABSTRACT

The invention relates to an intervertebral disk strain-relief support as an implant between spinous processes of adjacent vertebral bodies, comprising a carrier element having an opening and having first retaining wings, and a central element that has second retaining wings and that can be inserted into the opening of the carrier element. In the implanted state, the retaining wings lie outside on the spinous processes of adjacent vertebral bodies, and the spinous processes can be supported on opposite pressure surfaces on the carrier element and/or on the central element. According to the invention, at least one exposed spring slot, which is oriented transversely to the sagittal direction of extension of the spinal column and which extends over the area between two opposite pressure surfaces, is arranged in at least one of the elements in such a way that a resilient bridge is formed between the spring slot and one of the pressure surfaces of the implant, wherein the resilient bridge is resilient when the spinous processes are loaded.

### CROSS REFERENCE TO RELATED APPLICATIONS

This application is a United States National Phase application of International Application PCT/EP2010/050420 and claims the benefit of priority under 35 U.S.C. §119 of Swiss Patent Application 91/2009 filed Jan. 21, 2009, the entire contents of which are incorporated herein by reference.

### FIELD OF THE INVENTION

The invention relates to an intervertebral disc strain-relief support as an implant between spinous processes of adjacent vertebral bodies, comprising a carrier element with an opening and with first retaining wings, as well as a central element that has second retaining wings and can be inserted into the opening of the carrier element, the retaining wings, in the implanted state, resting outside on the spinous processes of adjacent vertebral bodies and the spinous processes being supported on opposing pressure surfaces on the carrier element and/or on the central element.

### BACKGROUND OF THE INVENTION

An implant for treating lumbar spinal canal stenosis with a spacer of adjacent spinous processes is known from WO 2006/064356. This is an implant substantially consisting of two components. The first component is inserted laterally between two spinous processes and the second component is then inserted into an opening provided for this of the first component. The first component has two folded-in wings, which are automatically folded out upon the insertion of the second component. Once the components have been joined together, they are fixed to one another, for example screwed. Together with two further wings, which are rigidly provided on the second component, a system is produced which is held rigidly between two spinous processes. The first and/or the second component forms a transversely extending roller body of the implant to relieve the intervertebral disc in that it forms a spacer between two adjacent spinous processes.

Implants of this type have proven successful in practice. The only disadvantage is that the roller body is absolutely rigid.

Various producers have attempted to use flexible materials, in particular plastics materials as the materials for the roller body. However, as these materials are unsatisfactory with the forces occurring here and have been little tested with regard to the long term behavior, they represent a certain risk.

### SUMMARY OF THE INVENTION

An object of the present invention is to disclose an implant of the type mentioned at the outset, which allows a certain flexibility, using tested metal alloys permitted in medical technology, while avoiding plastics materials.

The object is achieved by at least one exposed spring slot, which is oriented transversely to the sagittal running direction of the spinal column, and extends over the region between two opposing pressure surfaces, being arranged in at least one of the elements in such a way that a resilient bridge is formed between the spring slot and one of the pressure surfaces.

The idea on which the invention is based is that at least one exposed spring slot, which extends transversely to the sagittal running direction of the spinal column, is arranged between two opposing pressure surfaces in at least one of the elements in such a way that a resilient bridge having a resilient effect upon loading of the spinous processes is formed thereby between the spring slot and one of the pressure surfaces of the implant.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a side view of an implant arranged between two adjacent spinous processes of a vertebral body, in its installed position;

FIG. 2a is a first perspective view of an implant according to the invention in the assembled state;

FIG. 2b is a second perspective view of an implant according to the invention in the assembled state;

FIG. 2c is a third perspective view of an implant according to the invention in the assembled state;

FIG. 2d is a fourth perspective view of an implant according to the invention in the assembled state;

FIG. 3a is a schematic view of the carrier element and central element in a semi disassembled state viewed from above; and

FIG. 3b is a schematic view of the carrier element and central element in a partially disassembled state viewed from the side.

### DESCRIPTION OF THE PREFERRED EMBODIMENTS

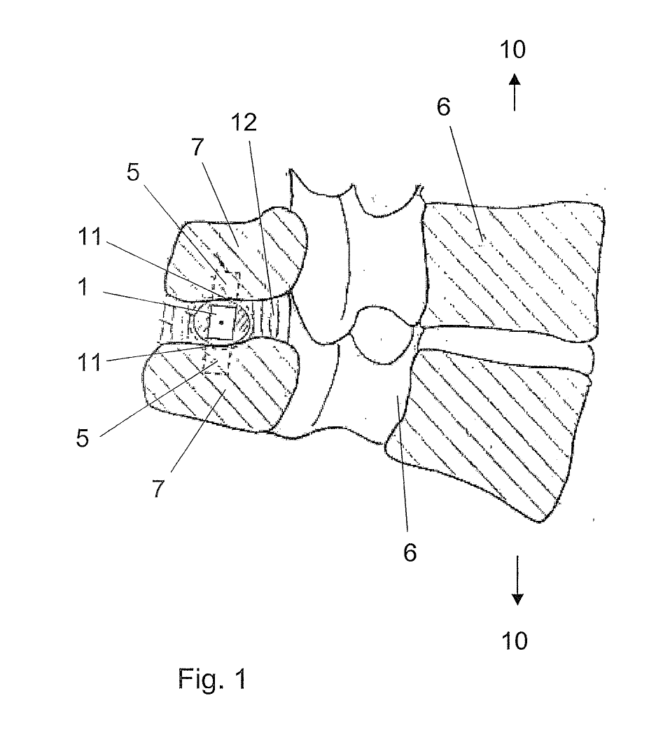

A part of the spinal column 10 of a patient is shown in FIG. 1a, with two adjacent vertebral bodies 6 and their spinous processes 7. The spinal column 10 extends in the direction of the arrows in this view. The interspinous ligament 12 runs between two adjacent spinous processes 7. Said interspinous ligament is crossed by an intervertebral disc strain-relief support 1 and held in its position on both sides of both adjacent spinous processes 7 by two retaining wing pairs 5, 5′ against transverse displacements. The intervertebral disc strain-relief support 1 is preferably configured tapering centrally to support the spinous processes 7 and therefore subjected to self-centering, so that it is additionally secured against displacements.

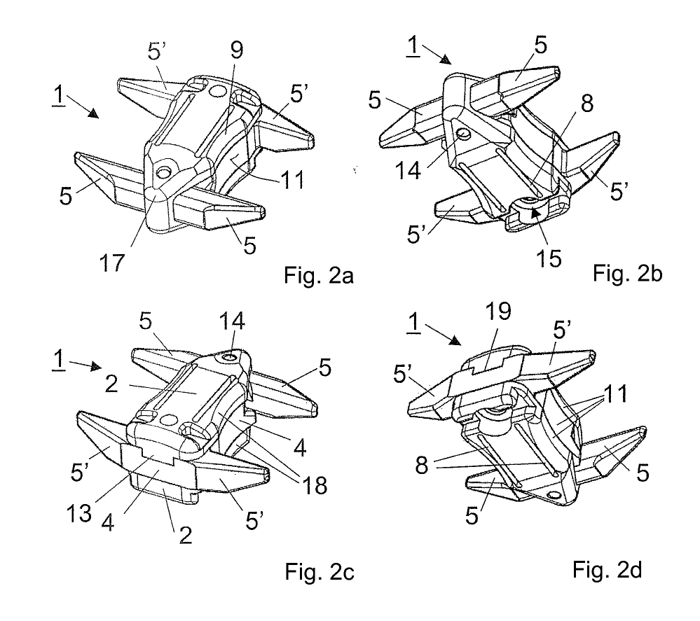

FIG. 2 shows the intervertebral disc strain-relief support 1 according to the invention in four different perspectives and in a fully assembled state. The four retaining wings 5, 5′, in the inserted state, in each case, rest on spinous processes 7, not shown here.

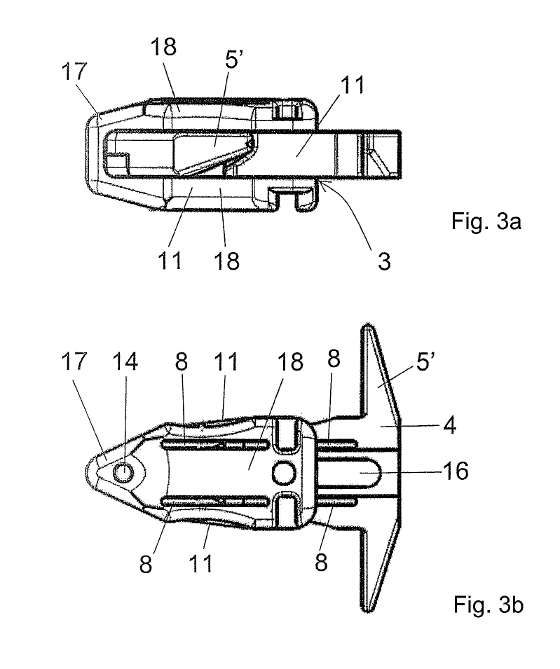

FIG. 3 a) and b) show schematic views of the intervertebral disc strain-relief support 1 viewed from above (a) and viewed from the side (b), in each case in partially disassembled states. The movable retaining wings 5′ on the central element 4, which can move about the rotational axis 14, are still located here between the support walls 18 of the carrier element 2.

The intervertebral disc strain-relief support 1 substantially consists of two components, namely of a carrier element 2 with an opening 3 and of a central element 4, which can be fittingly inserted in the opening 3 of the carrier element 2.

The carrier element 2 is organized, owing to the opening 3, into two parallel support walls 18, which are connected to one another in one piece in the region of an introduction head 17. Present in the region of the introduction head 17 is a rotational axis, about which the movable wings 5 can pivot. The wings lie between the two support walls 18 in a first phase of installation. This allows the lateral insertion of the carrier element 2 between two adjacent vertebral body processes. As a result, the central element 4 with its rigid wings 5′ can be inserted from the same side into the opening 3. This is facilitated by a rail 13 which is attached on the inside of one of the two support walls 18 and extends from the open end of the carrier element 2 to the introduction head 17 thereof, and through a mirror-inverted groove 19 in the central element 4.

When the central element 4 is inserted into the opening 3 of the carrier element 2, the folded-in retaining wings 5 are finally automatically moved out into the end position. A screw 15 finally fixes the two elements 2, 4 and firmly holds their relative position with respect to one another. The central element 4 preferably has an elongate hole 16, so that the relative position between the central element 4 and carrier element can be adapted to the widths of the spinous processes of the patient.

In the implanted state, the spinous processes 7 are supported on opposing pressure surfaces 11 on the carrier element 2 and/or on the central element 4. Intervertebral disc strain-relief supports described here are known from WO 2006/064356, which corresponds to US 2009/0254185 A1, the entire contents of which are incorporated herein by reference.

The intervertebral disc strain-relief supports 1 according to the invention shown in the figures, in addition to the intervertebral disc strain-relief supports 1 according to the prior art, have at least one exposed spring slot 8, which extends transversely to the sagittal running direction of the spinal column 10. The spring slot 8 is located in parallel to and between the two opposing pressure surfaces 11 in at least one of the elements 2, 4. The spring slot 8 is arranged in such a way that a resilient bridge 9 is formed between it and the next pressure surface 11. When the spinous processes 7 are loaded, this bridge 9 yields resiliently, so the desired flexibility is achieved. The spring slot 8 is preferably arranged close to a loadable pressure surface 11, so that the desired spring action is produced.

According to the prior art, a central slot in the central element 4 is formed as an elongate hole 16. This ensures that a screw 15 can ensure an adjustable spacing of the retaining wing pairs 5, 5′ during the connection of the two elements 2, 4, in accordance with the widths of the spinous processes 7 of the patient. This elongate hole 16 does not, however, fulfill the function of a spring slot 8 but optionally supports the action thereof.

The spring slot 8 according to the invention is provided close to a pressure surface 11, so that a thin resilient bridge 9 can be formed. The thinner the bridge 9, i.e. the smaller the distance between a pressure surface 11 and the spring slots 8 allocated thereto, the more yielding is the intervertebral disc strain-relief support 1.

Two opposing bridges 9, each being allocated to a spinous process 7, are preferably formed by at least two spring slots 8. Spring slots 8 of this type are to be provided on the element or on the elements 2, 4, on which the spinous processes 7 rest on pressure surfaces 11. If the pressure is distributed to the two side walls 18, a total of four spring slots 8 should be provided, two on each side wall 18, each allocated to one spinous process 7. If the pressure is additionally distributed to the central element, a total of six spring slots 8 are provided, the four described on the side walls 18 and two on the central element, one in the case of each spinous process.

Spring slots 8 should preferably be provided close to all the support surfaces 11, so that an optimal resilience is achieved. Depending on the configuration of the heights of the carrier element 2 and central element 4, a different number of spring slots 8 are to be provided, in particular 2, 4 or 6.

If the height of the elements 2, 4 is unequal, so that pressure surfaces 11 for the spinous processes 7 are only provided on one of the elements 2, 4 or on one part of the carrier element 2, two spring slots 8 on either side on this element 2 or 4 are sufficient. However, if the carrier element 2 and the central element 4 are the same height, so that each spinous process 7 has three support surfaces 11, a total of six spring slots 8 should be provided on the intervertebral disc strain-relief support 1, namely on both sides of the opening 3 of the carrier element 2 and on the central element 4, in each case one in the region of each spinous process 7.

Each pressure surface 11 of each element 2, 4 is to be formed by a spring slot 8, in each case, into a resilient bridge 9, so that, in the implanted state, the two spinous processes 7 rest completely cushioned on the pressure surfaces 11. The spring slots 8 should preferably substantially comprise the entire region of the elements 2, 4 between the two pairs of retaining wings 5, 5′, so the spring action of the bridge 9 is also ensured upon loading.

In principal, the spring slots 8 do not have to run in parallel on both sides on the supporting walls 18 nor at the same spacings from the pressure surfaces 11. However, this may lead to the spring action of the bridges 9 being different, so that shear movements may occur. This is undesired and therefore the spring slots 8 are arranged the same and running in parallel with respect to the size and the running direction in the two support walls and in the carrier element, so that in the assembled state, all the spring slots 8 lying close to a spinous process 7 lie congruently one above the other.

Although it would be sufficient to arrange one spring slot 8 in each case in the two support walls and the carrier element 2, allocated to only one spinous process, the version shown in the drawings with an arrangement of the spring slots 8 on both sides is preferably implemented.

While specific embodiments of the invention have been described in detail to illustrate the application of the principles of the invention, it will be understood that the invention may be embodied otherwise without departing from such principles.

### LIST OF REFERENCE NUMERALS

1 intervertebral disc strain-relief support
2 carrier element
3 opening
4 central element
5,5′ retaining wings
6 vertebral body
7 spinous process
8 spring slot
9 resilient bridge
10 spinal column
11 pressure surface
12 ligament
13 rail
14 rotational axis
15 screw
16 elongate hole
17 introduction head
18 support walls of the carrier element
19 groove

1. An intervertebral disc strain-relief support as an implant between spinous processes of adjacent vertebral bodies, comprising:
a carrier element with an opening and with first retaining wings; and
a central element that has second retaining wings and is insertable into the opening of the carrier element, wherein, in the implanted state, the retaining wings rest outside on the spinous processes of adjacent vertebral bodies and the spinous processes is supportable on opposing pressure surfaces on at least one of the carrier element and the central element, wherein at least one exposed spring slot, which is oriented transversely to the sagittal running direction of the spinal column, and extends over the region between two opposing pressure surfaces, is arranged in at least one of the elements in such a way that a resilient bridge is formed between the spring slot and one of the pressure surfaces.

2. An intervertebral disc strain-relief support according to claim 1, wherein two bridges, which are each allocated to a spinous process, are formed by at least two spring slots.

3. An intervertebral disc strain-relief support according to claim 1, wherein each pressure surface of each element is formed by a spring slot into a bridge, in each case, so that in the implanted state, the two spinous processes rest on the resilient pressure surfaces.

4. An intervertebral disc strain-relief support according to claim 1, wherein the spring slots extend substantially over the entire region between the pressure surfaces.

5. An intervertebral disc strain-relief support according to claim 1, wherein the carrier element has two parallel support walls, which are connected to one another by means of an introduction head and in each of which two spring slots are provided.

6. An intervertebral disc strain-relief support according to claim 5, wherein the central element has two spring slots.

7. An intervertebral disc strain-relief support according to claim 1, wherein all the bridges up to the spring slots have the same thickness.

8. An intervertebral disc strain-relief support according to claim 1, wherein all the spring slots extend parallel to one another and all the spring slots, which are allocated to the same pressure surface, lie aligned one behind the other.
